(19) United States
(12) Patent Application Publication
(10) Pub. No.: US 2010/0004267 A1
(43) Pub. Date: Jan. 7, 2010

(54) LIQUID PREPARATION

(75) Inventors: Hiroo Kawamura, Tochigi (JP); Tetsu Ono, Osaka (JP)

Correspondence Address: DRINKER BIDDLE & REATH (DC), 1500 K STREET, N.W., SUITE 1100, WASHINGTON, DC, 20005-1209, US

(73) Assignee: ASUBIO PHARMA CO., LTD., Minato-ku (JP)

(21) Appl. No.: 12/375,127

(22) PCT Filed: Jul. 31, 2007
(86) PCT No.: PCT/JP2007/065326

§ 371 Date: Aug. 11, 2009

(30) Foreign Application Priority Data
Jul. 31, 2006 (JP) 2006-209305

(51) Int. Cl. A61K 31/4965; A61P 9/10

(52) U.S. Cl. 514/255.03

(57) ABSTRACT

It is provided a liquid preparation stably including (2S)-1-(4-amino-2,3,5-trimethylphenoxy)-3-{4-[4-(4-fluorobenzyl)phenyl]-1-piperazinyl}-2-propanol or its pharmaceutically acceptable salt as an active ingredient.

The present invention relates to a liquid preparation including the above compound or its pharmaceutically acceptable salt as an active ingredient, the liquid preparation including:
(a) at least one selected from the group consisting of sulfite, bisulfite, pyrosulfite, α-thioglycerol and cysteine,
(b) a β-cyclodextrin derivative, and
(c) a pH buffer,
   wherein the pH value thereof is adjusted within a range of from 3 to 5.

### TECHNICAL FIELD

The present invention relates to a liquid preparation which contains, as its active ingredient, (2S)-1-(4-amino-2,3,5-trimethylphenoxy)-3-{4-[4-(4-fluorobenzyl)phenyl]-1-piperazinyl}-2-propanol or its pharmaceutically acceptable salt, having remedial and therapeutic effect against symptoms based on ischemic disorder and neurodegenerative disease, symptoms from spasm, epilepsy and migraine, as well as various symptoms caused by diabetes, arteriosclerosis and inflammatory disease.

### BACKGROUND ART

(2S)-1-(4-amino-2,3,5-trimethylphenoxy)-3-{4-[4-(4-fluorobenzyl)phenyl]-1-piperazinyl}-2-propanol shown by the formula (I):

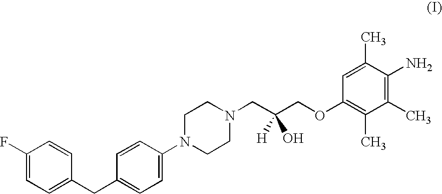

or its pharmaceutically acceptable salt is a compound having remedial and therapeutic effect against symptoms based on ischemic disorder and neurodegenerative disease, symptoms from spasm, epilepsy and migraine, as well as various symptoms caused by diabetes, arteriosclerosis and inflammatory disease because it has not only an ability to block Na⁺ and T-type Ca²⁺ channel of neuron, but also anti-oxidization activity. In particular, a dimethane sulfonic acid salt thereof is a clinically interested compound (Patent document 1 and Non-patent document 1).

This compound and its pharmaceutically acceptable salt have very poor solubility at pH around neutrality, and do not have good stability in-solution. In consideration of clinically expected dose, and desirable preparation form of the compound, solubility of 5 mg/mL or higher is required. However, since solubility at pH around neutrality is very low, it is difficult to develop an injectable preparation by usual formulation techniques.

In developing an injectable preparation, it is necessary to ensure stability of contained active ingredients during production or storage, as well as to avoid stimulus property of the preparation at the time of administration. In particular, for use as a therapeutic drug against acute ischemic disorder for which emergent administration of drug is required, there is a need of developing a liquid preparation, which serves as an injectable preparation stably containing the above compound and its pharmaceutically acceptable salt.
[Patent document 1] International Patent Publication WO99/23072
[Non-patent document 1] J. Med. Chem., 2000, 43, 3372-3376

### DISCLOSURE OF THE INVENTION

### [Problems to be Solved by the Invention]

Therefore, it is an object of the present invention to provide a liquid preparation, which stably contains a compound represented by the abovementioned formula (I) or its pharmaceutically acceptable salt as an active ingredient.

In order to solve the problem, the inventors of the present invention made diligent efforts and newly found that a stable liquid preparation is obtained by mixing the compound represented by the abovementioned formula (I) or its pharmaceutically acceptable salt and an antioxidant, with a specific solubilizing agent comprising a β-cyclodextrin derivative, and adjusting pH of the resultant solution within a specific range, and accomplished the present invention.

### [Means for Solving the Problem]

Therefore, the present invention provides as basic aspects,
(1) a liquid preparation comprising, as an active ingredient, (2S)-1-(4-amino-2,3,5-trimethylphenoxy)-3-{4-[4-(4-fluorobenzyl)phenyl]-1-piperazinyl}-2-propanol represented by the formula (I) or its pharmaceutically acceptable salt, the preparation further comprising:

(a) at least one selected from the group consisting of sulfite, bisulfite, pyrosulfite, α-thioglycerol and cysteine;

(b) a β-cyclodextrin derivative, and

(c) a pH buffer,

wherein the pH value thereof is adjusted to fall within a range of from 3 to 5.

More specifically, the present invention provides:
(2) The liquid preparation according to the above (1) comprising (a) at least one selected from the group consisting of sodium bisulfite, sodium pyrosulfite, α-thioglycerol, L-cysteine hydrochloride monohydrate and L-cysteine;
(3) The liquid preparation according to the above (1) comprising (a) sodium bisulfite;
(4) The liquid preparation according to the above (1) comprising (a) sodium bisulfite and L-cysteine hydrochloride monohydrate;
(5) The liquid preparation according to the above (4), wherein a quantity ratio of sodium bisulfite and L-cysteine hydrochloride monohydrate is between 1/10 and 1/3 by L-cysteine hydrochloride monohydrate/sodium bisulfite (w/w);
(6) The liquid preparation of any one of the above (3) to (5), wherein a concentration of sodium bisulfite is in a range of from 0.02 to 0.30% (w/v);
(7) The liquid preparation of any one of the above (3) to (6), wherein a quantity of sodium bisulfite is 1/35 to 3 times (weight ratio) relative to the active ingredient;
(8) The liquid preparation according to the above (1), wherein the β-cyclodextrin derivative is β-cyclodextrin sulfobutyl ether sodium salt or hydroxypropyl β-cyclodextrin;
(9) The liquid preparation according to the above (1) or (8), wherein a quantity of the β-cyclodextrin derivative relative to the active ingredient is such that a ratio between active ingredient: β-cyclodextrin derivative falls between 1:4 and 1:8 (molar ratio);
(10) The liquid preparation according to the above (1) or (8), wherein a concentration of the β-cyclodextrin derivative is in a range of from 7.5 to 13.5% (w/v);
(11) The liquid preparation according to the above (1), wherein the pH buffer is of amino acid buffer system or acetic acid buffer system;
(12) The liquid preparation according to the above (11), wherein the amino acid buffer system comprises glycine or L-histidine;
(13) The liquid preparation according to the above (12), wherein the amino acid buffer system comprises glycine with a concentration thereof in a range of from 50 to 150 mM;
(14) The liquid preparation according to the above (1), wherein the pH value thereof is adjusted with a pH adjuster, which is at least one selected from the group consisting of hydrochloric acid, methanesulfonic acid, acetic acid and sodium hydroxide;
(15) The liquid preparation according to the above (14), wherein the pH adjuster is hydrochloric acid;
(16) The liquid preparation according to the above (1) which is prepared using water in which dissolved oxygen is reduced by inert gas replacement;
(17) The liquid preparation according to the above (1), wherein a concentration of dissolved oxygen is less than or equal to 4 ppm;
(18) The liquid preparation according to the above (1), further comprising a substance that reduces surface activity derived from the active ingredient;
(19) The liquid preparation according to the above (18), wherein the substance that reduces the surface activity is at least one selected from the group consisting of sodium chloride, meglumine, L-arginine, glycine, L-histidine, L-glutamate, β-cyclodextrin derivative, propylene glycol, ethanol and chlorobutanol;
(20) The liquid preparation according to the above (1), further comprising a substance that regulates formation of insoluble aggregates with free base of (2S)-1-(4-amino-2,3,5-trimethyl-phenoxy)-3-{4-[4-(4-fluorobenzyl)phenyl]-1-piperazinyl}-2-propanol; and
(21) The liquid preparation according to the above (20), wherein the substance that regulates formation of insoluble aggregates is at least one selected from the group consisting of methanesulfonic acid, hydrochloric acid and sodium chloride.

The most specific invention is:
(22) A liquid preparation comprising: 1 to 5 mg/mL of (2S)-1-(4-amino-2,3,5-trimethylphenoxy)-3-{4-[4-(4-fluorobenzyl)phenyl]-1-piperazinyl}-2-propanol or its pharmaceutically acceptable salt; 0.05 to 0.10% (w/v) of sodium bisulfite; 0.015 to 0.03% (w/v) of L-cysteine hydrochloride monohydrate; 7.5 to 10% (w/v) of β-cyclodextrin sulfobutyl ether sodium salt (representatively, 7 sodium salt); 50 to 100 mM of glycine; and hydrochloric acid, wherein the pH value thereof falls within a range of from 3.0 to 4.5.

Also, the present invention provides:
(23) A liquid preparation comprising the liquid preparation according to any one of the above (1) to (22) filled into a glass container having an inner wall treated with SiO₂ glass film at the liquid contacting side.

In another aspect, the present invention provides a method of producing the foregoing liquid preparation, more specifically, a method of producing a liquid preparation comprising the steps of:

(a) dissolving (2S)-1-(4-amino-2,3,5-trimethylphenoxy)-3-{4-[4-(4-fluorobenzyl)phenyl]-1-piperazinyl}-2-propanol or its pharmaceutically acceptable salt into water in which dissolved oxygen is reduced by inert gas replacement to prepare a solution of drug substance;

(b) dissolving a β-cyclodextrin derivative, a pH buffer and a pH adjuster into water and adjusting the pH value thereof to fall within a range of from 3 to 5, replacing dissolved oxygen by inert gas replacement, and dissolving at least one selected from the group consisting of sulfite, bisulfite, pyrosulfite, α-thioglycerol and cysteine to prepare a solution of inactive ingredients; and

(c) adding the solution of drug substance to the solution of inactive ingredients under inert gas flow and mixing the same.

### [Advantage of the Invention]

According to the present invention, there is provided a stable liquid preparation which comprises, as an active ingredient, a compound represented by the formula (I) or its pharmaceutically acceptable salt having very poor solubility at pH around neutrality.

The liquid preparation provided by the present invention stably comprises a compound represented by the formula (I) and its pharmaceutically acceptable salt which is an active ingredient, and since such compound and its pharmaceutically acceptable salt have excellent remedial and therapeutic ability against symptoms based on ischemic disorder and neurodegenerative disease, symptoms from spasm, epilepsy and migraine, as well as various symptoms caused by diabetes, arteriosclerosis and inflammatory disease, the liquid preparation is highly effective for therapy of these diseases.

### [Best Mode for Carrying out the Invention]

As described above, a basic aspect of the present invention is a liquid preparation comprising, as an active ingredient, (2S)-1-(4-amino-2,3,5-trimethylphenoxy)-3-{4-[4-(4-fluorobenzyl)phenyl]-1-piperazinyl}-2-propanol represented by the formula (I) or its pharmaceutically acceptable salt, the preparation further comprising:

(a) at least one selected from the group consisting of sulfite, bisulfite, pyrosulfite, α-thioglycerol and cysteine;

(b) a β-cyclodextrin derivative, and

(c) a pH buffer,

wherein the pH value thereof is adjusted to fall within a range of from 3 to 5.

As for a pharmaceutically acceptable salt of the compound represented by the formula (I), treatment of the compound of formula (I) which is free base, together with an inorganic acid or organic acid in a suitable solvent will give a corresponding salt. Examples of such inorganic acid include hydrochloric acid, hydrobromic acid, sulfuric acid, nitric acid, phosphoric acid, periodic acid and the like inorganic acids. Examples of organic acid include formic acid, acetic acid, butyric acid, oxalic acid, malonic acid, propionic acid, valeric acid, succinic acid, fumaric acid, maleic acid, tartaric acid, citric acid, malic acid, benzoic acid, p-toluene sulfonic acid, methane sulfonic acid, ethane sulfonic acid, trifluoromethane sulfonic acid, benzene sulfonic acid and the like organic acids.

In this case, a salt comprising one to three acid molecules may be selectively produced by increasing/decreasing the use amount of inorganic or organic acid within the range of 1 to 3 equivalents, depending on the number of basic nitrogen atom in the compound of formula (I).

In the present invention, dimethane sulfonic acid salt is particularly preferred as the pharmaceutically acceptable salt of formula (I). This compound, namely (2S)-1-(4-amino-2,3,5-trimethylphenoxy)-3-{4-[4-(4-fluorobenzyl)phenyl]-1-piperazinyl}-2-propanol dimethane sulfonate is a compound for which clinical development is being examined under the development code number: SUN N8075.

Therefore, in the following explanation, as the compound represented by the formula (I) or its pharmaceutically acceptable salt, (2S)-1-(4-amino-2,3,5-trimethylphenoxy)-3-{4-[4-(4-fluorobenzyl)phenyl]-1-piperazinyl}-2-propanol dimethane sulfonate (hereinafter, simply referred to “SUN N8075”) will be described as a representative example.

The present invention will be explained more specifically by sequentially explaining the practice of formulation of SUN N8075 and formulation consideration by way of Test examples instead of Examples.

It is to be noted that these Test examples are given for understanding of the present invention, and the scope of the present will not be limited by these Test examples.

In a liquid preparation provided by the present invention, a compound of the formula (I) represented by SUN N8075, or its pharmaceutically acceptable salt contained as an active ingredient has very low solubility, and an aqueous solution of the compound has properties such as stimulus property when administered as an injectable preparation, self-oxidation property, low stability against light and susceptibility to light decomposition. Furthermore, the present compound in its aqueous solution probably has the basic property that it is likely to generate insoluble matters due to the chemical changes, physiochemical changes, interfacial chemical changes such as self oxidation, association with other chemical species, self aggregation and the like, which are extremely disadvantageous properties in making a liquid preparation.

Therefore, the problem to be considered in the present formulation is to ensure sufficient solubility as a liquid preparation, to reduce stimulus when administered to blood vessel, to regulate oxidation and light decomposition of active ingredient by storage, and to prevent generation of insoluble matters. Also, it is necessary to consider about prevention of generation of insoluble matters (glass flakes) from a glass container such as glass ampule or vial.

In the following, examinations for these points will be explained sequentially.

### TEST EXAMPLE 1

### Examination of Solubilizing Agent (Part 1)

The present inventors examined properties of SUN N8075 itself, and proved that it had very low solubility at pH around neutrality.

Therefore, in order to examine a liquid preparation containing SUN N8075 as an active ingredient, it is inevitable to add a solubilizing agent that improves solubility of the resultant liquid preparation.

In view of this, solubility of SUN N8075 was examined using compounds listed in Table 1 below which are conventionally used as a solubilizing agent.

### [Method]

8 mg/mL aqueous solution of SUN N8075 was prepared. On the other hand, for each solubilizing agent, an aqueous solution having approximately 0.4% of solubilizing agent was prepared.

10 mL of SUN N8075 aqueous solution and 10 mL of solution of each solubilizing agent were mixed to give Mixture A, and Mixture A was filtered through a filter of 0.22 μm.

6.24 g of sodium dihydrogen phosphate and 16.4 g of sodium chloride were weighed and water was added to make the volume precisely 1,000 mL. To this solution, 1 mol/L sodium hydroxide test solution was added dropwise and the pH value was adjusted to 7.4, which was named Mixture B.

Mixture of each 2.5 mL of Mixture A and Mixture B was provided as a test solution (in each test solution, the concentration of SUN N8075 corresponds to 2 mg/mL, and the concentration of solubilizing agent corresponds to 0.1%).

The test solution was shaken in a reciprocating shaker at 24° C. for 30 minutes, filtered through a filter of 0.22 μm, and the concentration of SUN N8075 in the filtrate was measured by HPLC method to determine its solubility.

As for a solubilizing agent of which degree of improving solubility of SUN N8075 was high, tests with concentrations of the added solubilizing agent varied were additionally executed in accordance with the method as described above.

### [Results]

The results are collectively shown in Table 1.

TABLE 1

| | Adding | | Appearance | |
|---|---|---|---|---|
| | Conc. | | of | Concentration |
| Solubilizing agent | (%) | pH | solution | (μg/mL) |
| No additive | — | 6.8 | colorless and clear | ND |
| Glycerin | 0.1 | 6.8 | colorless and clear | ND |
| Macrogol 4000*¹ | 0.1 | 6.8 | colorless and clear | ND |
| Mannitol | 0.1 | 6.8 | colorless and clear | ND |
| Ethylene diamine*⁵ | 0.1 | — | — | — |
| Nicotinic amide | 0.1 | 6.8 | colorless and clear | ND |
| Propylene glycol | 0.1 | 6.9 | colorless and clear | ND |
| Meglumine*⁵ | 0.1 | — | — | — |
| β-Cyclodextrin | 0.1 | 6.9 | colorless and clear | 26 |
| Human serum | 0.1 | 6.8 | colorless and clear | 14 |
| albumin | | | | |
| HCO-60*² | 0.1 | 6.8 | colorless and clear | 184 |
| | 0.2 | 6.9 | colorless and clear | 334 |
| | 0.5 | 6.8 | colorless and clear | 395 |
| Tween80*³ | 0.1 | 6.8 | clouded | 745 |
| | 0.2 | 6.8 | clouded | 1830 |
| | 0.5 | 6.8 | clouded | 1790 |
| | 1.0 | 6.8 | colorless and clear | 1820 |
| β-Cyclodextrin | 0.1 | 6.8 | colorless and clear | ND |
| derivative*⁴ | 0.5 | 6.8 | colorless and clear | 98 |
| | 1.0 | 6.8 | colorless and clear | 240 |
| | 5.0 | 6.9 | colorless and clear | 1425 |
| | 10 | 6.5 | colorless and clear | 4851 |

*¹polyethylene glycol 4000
*²polyoxyethylene hardened castor oil
*³polyoxyethylene (20) sorbitan monooleate
*⁴β-cyclodextrin sulfobutyl ether sodium salt
*⁵Test was halted because solubilizing agent solution clouded
ND: Not detected

From these examinations, it was found that polyoxyethylene (20) sorbitan monooleate (Tween80) and β-cyclodextrin sulfobutyl ether sodium salt, which is a β-cyclodextrin derivative, are effective as a solubilizing agent.

### TEST EXAMPLE 2

### Examination of Solubilizing Agent (Part 2)

The objective preparation of the present invention is a liquid preparation, which is mainly administered in the form of an injectable preparation. Therefore, it is requested that there is no stimulus in tissues at the site of administration at the time of administration.

Stimulus property in administration was examined for polyoxyethylene (20) sorbitan monooleate (Tween80) and β-cyclodextrin sulfobutyl ether sodium salt, which is a β-cyclodextrin derivative, which were found, as a result of the examination of Test example 1, to be an effective solubilizing agent for improvement of solubility of SUN N8075.

### [Method]

Saline, a solution in saline to which 0.1% of Tween80 was added, and a solution in saline to which 0.5, 1.0, 2.0 or 3.0% of β-cyclodextrin sulfobutyl ether sodium salt dissolving various concentrations described in Table 2 below of SUN N8075 were prepared, and administered to aorta of rat, and precipitation of SUN N8075 and presence of stimulus (blood vessel stimulus) on tissues at the site of administration were evaluated.

### [Results]

These results are collectively shown in Table 2.

TABLE 2
Blood vessel stimulus by SUN N8075 and stimulus suppressing
effect by β-cyclodextrin sulfobutyl ether sodium salt
[hereinafter referred to as Captisol (registered trademark)] (rat)

| Solubilizing agent | Drug | Administ. | Precipitation at | |
|---|---|---|---|---|
| concentration | conc. | Time | administration | Blood vessel |
| In saline | (mg/mL) | (hour) | site | stimulation |
| None | 0.23 | 72 | Detected*¹ | Detected |
| | 0.7 | 72 | Detected*¹ | Detected |
| | 2.3 | 72 | Detected*¹ | Detected |
| 0.1% Tween80 | 0.23 | 72 | None*² | Detected |
| | 0.7 | 72 | None*² | Detected |
| 3.0% Captisol | 0.23 | 72 | None*² | None |
| | 0.7 | 72 | None*² | None |
| 2.0% Captisol | 0.7 | 24 | None*² | None |
| 1.0% Captisol | 0.7 | 24 | None*² | None |
| 0.5% Captisol | 0.7 | 24 | None*² | None |

*¹Observed on polarization microscope
*²Finding by naked eyes at autopsy and result of pathological examination

From these results, as to the administration solution in which Captisol which is a β-cyclodextrin derivative was added as a solubilizing agent, precipitation of crystals of SUN N8075 was not observed at the site of administration, and no blood vessel stimulus was observed.

β-cyclodextrin sulfobutyl ether sodium salt, which is a β-cyclodextrin derivative, is commercially available from CyDex Inc. in U.S. in the name of “Captisol (registered trademark)”.

A β-cyclodextrin derivative which may be used in the present invention has improved water solubility compared to β-cyclodextrin by introducing a substituent such as a sulfoalkyl group or hydroxyalkyl group into β-cyclodextrin, and hydroxypropyl β-cyclodextrin can be exemplified in addition to the aforementioned β-cyclodextrin sulfobutyl ether sodium salt. Products having various average degrees of substitution are commercially available. Among others, those having degree of substitution by sulfobutyl group is about 7 are preferred, and Captisol (registered trademark) is exemplified as such a product.

It was also found that the use amount with respect to the active ingredient is preferably 1:4 to 1:8 (molar ratio) as a ratio of active ingredient: β-cyclodextrin derivative, and additionally, the concentration of β-cyclodextrin derivative is preferably 7.5-13.5% (w/v).

### TEST EXAMPLE 3

### Examination of Antioxidant

In the liquid preparation provided by the present invention, the compound of the formula (I) represented by SUN N8075 or its pharmaceutically acceptable salt, which is an active ingredient, is easily oxidized.

Therefore, it is necessary to prevent oxidation of SUN N8075 dissolved in an aqueous solution as a liquid preparation.

Accordingly, various antioxidants were examined for antioxidant effect against SUN N8075.

### [Method]

In this examination, hydrochloride of the compound of formula (I) was used instead of SUN N8075. Approximately 400 mg of the hydrochloride of compound of formula (I) were precisely weighed and water was added to make the volume precisely 200 mL, which was provided as an original drug aqueous solution.

Approximately 400 mg of each antioxidant shown in Table 3 below were precisely weighed and dissolved by adding 20 mL of water. As to butylhydroxy anisole, 10.7 mg was precisely weighed and dissolved in 100 mL of water.

15 mL of each antioxidant solution was mixed with 15 mL of solution of drug substance to prepare a stock sample (in the sample, the concentration of the hydrochloride compound of formula (I) corresponds to 1 mg/mL and the concentration of antioxidant corresponds to 10 mg/mL).

After dispensing each stock sample into 15 brown glass ampules, the ampules were sealed and stored at 60° C.

As a control sample, 15 mL of solution of drug substance added with 15 mL of water were stored in the same condition.

The remaining three ampules for each sample solution were used for measurement at the start of tests.

Each prepared sample was sampled in time series. 1 mL of sample solution was precisely measured, change in appearance and solution state of the sample were observed, and then added with methanol to make the volume precisely 10 mL. For this solution, residual percentage of the hydrochloride of compound of formula (I) was calculated by HPLC method. Furthermore, the pH value of the sample solution remaining after completion of test was measured.

### [Results]

These results are collectively shown in Table 3 below.

TABLE 3

| | | | | Residual | |
|---|---|---|---|---|---|
| | Storage | | | degree | Purity |
| Antioxidant | period | Appearance | pH | (%) | (%) |
| No additive | At start | colorless and clear | 2.8 | 100 | 100 |
| | 1 day | colorless and clear | 2.8 | 98.7 | 98.4 |
| | 7 days | colorless and clear | 3.0 | 84.4 | 82.5 |
| | 21 days | colorless and clear | 2.9 | 43.1 | 51.2 |
| Sodium | At start | colorless and clear | 3.5 | 100 | 97.2 |
| bisulfite | 1 day | colorless and clear | 3.2 | 99.9 | 98.5 |
| | 7 days | colorless and clear | 2.8 | 97.3 | 98.3 |
| | 21 days | colorless and clear | 2.8 | 91.2 | 98.4 |
| α-Thioglycerol | At start | colorless and clear | 2.7 | 100 | 100 |
| | 1 day | colorless and clear | 2.8 | 97.5 | 100 |
| | 7 days | colorless and clear | 2.8 | 97.1 | 99.2 |
| | 21 days | colorless and clear | 2.8 | 89.0 | 96.8 |
| L-Cysteine HCl | At start | colorless and clear | 1.7 | 100 | 100 |
| monohydrate | 1 day | colorless and clear | 1.7 | 97.9 | 100 |
| | 7 days | colorless and clear | 1.7 | 100.6 | 99.7 |
| | 21 days | colorless and clear | 1.7 | 94.0 | 99.6 |
| Sodium edetate | At start | colorless and clear | 4.0 | 100 | 100 |
| | 1 day | colorless and clear | 3.8 | 94.1 | 97.9 |
| | 7 days | colorless and clear | 4.1 | 68.5 | 81.2 |
| | 21 days | colorless and clear | 4.2 | 31.9 | 53.8 |
| Citric acid | At start | colorless and clear | 2.1 | 100 | 100 |
| | 1 day | colorless and clear | 2.2 | 98.0 | 99.2 |
| | 7 days | colorless and clear | 2.2 | 89.8 | 88.6 |
| | 21 days | colorless and clear | 2.2 | 57.3 | 63.6 |
| Sodium | At start | colorless and clear | 3.6 | 100 | 94.4 |
| pyrosulfite | 1 day | colorless and clear | 3.1 | 102.4 | 98.2 |
| | 7 days | colorless and clear | 2.9 | 97.2 | 97.3 |
| | 21 days | colorless and clear | 2.7 | 89.8 | 97.6 |
| Butyl hydroxy | At start | colorless and clear | 2.8 | 100 | 100 |
| anisole | 1 day | colorless and clear | 2.8 | 99.2 | 96.8 |
| | 7 days | colorless and clear | 2.8 | 80.3 | 80.6 |
| | 21 days | Brown precipitates | 2.8 | 41.2 | 53.5 |

From these results, it was found that sodium bisulfite, sodium pyrosulfite, L-cysteine hydrochloride monohydrate and α-thioglycerol provide desired anti-oxidation effect on the solution of SUN N8075. In addition to sodium bisulfite and sodium pyrosulfite, sulfites such as sodium sulfite, potassium sulfite and calcium sulfite, bisulfites such as potassium bisulfite and ammonium bisulfite, pyrosulfites such as potassium pyrosulfite and the like may be used, and in addition to L-cysteine hydrochloride monohydrate, cysteine such as L-cysteine, DL-cysteine and hydrochlorides thereof may also be used.

Among these, sodium bisulfite is particularly preferred from the viewpoint of the amount used as an ingredient in parenteral dosage form for clinical use as the experience.

These antioxidants may preferably be used singly or in combination, and more specifically, for example, sodium bisulfite and L-cysteine hydrochloride monohydrate may be used in combination. Also, equivalent mol of L-cysteine may be used instead of L-cysteine hydrochloride monohydrate.

When sodium bisulfite is used as an antioxidant, the concentration falls within the range of from 0.02 to 0.30% (w/v), and preferably from 0.05 to 0.10% (w/v), and the adding amount thereof is 1/35 to 3 times, and preferably 1/10 to 1/5 times (weight ratio) relative to the content of SUN N8075 which is the active ingredient.

When the concentration of sodium bisulfite is less than 0.02% (w/v), the desired anti oxidation effect is not exerted even in the case of administration, and the use of an amount exceeding 0.30% (w/v) will not give further effect and rather promote alkaline elution in a glass container, which may generate glass flakes.

Likewise, when the adding amount of sodium bisulfite is less than 1/35 times (weight ratio) relative to the content of SUN N8075 which is the active ingredient, desired anti-oxidation effect is not exerted, whereas when it is more than three times (weight ratio), glass flakes from the glass container may generate.

Furthermore, when sodium bisulfite and L-cysteine hydrochloride monohydrate are used in combination, the quantity ratio preferably satisfies L-cysteine hydrochloride monohydrate/sodium bisulfite (w/w) of from 1/10 to 1/3. That is, by adding L-cysteine hydrochloride monohydrate, it is preferred to reduce the adding amount of sodium bisulfite, and control generation of glass flakes caused by alkaline elution due to erosion of glass container by sulfites or bisulfites (for this point, a more specific description will be given later including regulation of glass flakes and other elements).

According to the examination made by the present inventors, it was found that addition of bisulfite salt, particularly sodium bisulfite as an antioxidant for SUN N8075 is preferred, however, sodium bisulfite is likely to erode glass. Therefore, there is a possibility that insoluble matters (glass flakes) from glass may generate during a long-term storage.

In order to avoid this point, according to the liquid preparation of the present invention, it is preferred to fill the liquid preparation into a glass container having subjected to surface coating for preventing generation of glass flakes.

As a glass container having such surface coating, for example, containers of sulpha-treated glass or glass coated with silicon polymer, or glass container having an inner wall treated with SiO₂ glass film can be recited. In the present invention, by using a glass container having an inner wall treated with SiO₂ glass film at the side where it contacts liquid, in particular, generation of glass flakes can be prevented.

The liquid preparation provided by the present invention is a solution preparation containing a compound represented by the formula (I) or its pharmaceutically acceptable salt, for example, SUN N8075, and is administered parenterally in the form of injectable preparation. The dose is not uniquely defined because it differs depending on various factors, including condition of the patient to be treated, degree of severity, age, presence/absence of complication and the like.

However, from the result of study for clinical use amount of SUN N8075, it was found that by preparing a liquid preparation containing 1 to 7 mg/mL, preferably 1 to 5 mg/mL of SUN N8075 as a liquid preparation and appropriately adjusting the dose, it can be used in therapy of target disease.

Among others, it is particularly preferred to prepare a liquid preparation containing 5 mg/mL of SUN N8075 and appropriately adjusting the dose for use in therapy of target disease.

Furthermore, since it is a liquid preparation which is administered by injection and the like administration route, the pH value of the liquid preparation itself is preferably adjusted within the range of from 3 to 5. This pH range is determined in consideration of solubility and stability of the compound represented by the formula (I) or its pharmaceutically acceptable salt, for example, SUN N8075. If the pH value is less than 3, it is not favorable for injection, and when the pH value exceeds 5, the stability of the liquid preparation itself is not favorable.

For adjustment of pH, various pH adjusters may be used, and as such pH adjuster, hydrochloric acid, methane sulfonic acid, sodium hydroxide and the like can be exemplified. Among these, hydrochloric acid is preferably used.

In the liquid preparation of the present invention for which the pH value is to be adjusted, it is preferred to use a pH buffer together with the pH adjuster in order to minimize any variation of the pH during storage.

As such a pH buffer, a variety of buffers based on amino acid, inorganic acid, and organic acid can be exemplified, and among these, an amino acid-based buffer or acetic acid buffer which is an organic acid-based buffer is particularly preferred.

As an amino acid that forms amino acid-based buffer, glycine or L-histidine is preferred, and glycine is particularly preferred. At this time, the concentration thereof may be selected to minimize the pH variation, and concretely, 30 to 150 mM, and preferably about 50 to 100 mM of glycine may be contained.

As an acetic acid buffer, acetic acid or combination of acetic acid and sodium acetate can be exemplified.

From results of these basic examinations described above, selection of a compound of the formula (I) represented by SUN N8075 or its pharmaceutically acceptable salt, a solubilizing agent therefore, in particular, β-cyclodextrin derivative, selection of an antioxidant, for example, sulfite, bisulfite, pyrosulfite, α-thioglycerol and cysteine, selection of a pH adjuster, and selection of a pH buffer allow preparation of an objective liquid preparation containing a compound represented by the formula (I) or its pharmaceutically acceptable salt, in particular, SUN N8075 and having its pH value ranging from 3 to 5.

In view of the above, the present inventors examined stability in long-term storage of a specific liquid preparation based on combination of these additives and studied for development of liquid preparation, which is suited for clinical application.

These points will be described below.

First, as to the liquid preparation provided by the present invention, the pH value of the solution is adjusted within the range of from 3 to 5, and variation of the pH value and its effect on the properties of the preparation should be as small as possible. In addition, since the liquid preparation is directly administered by injection or drip, insoluble matters should not be generated during storage. For this reason, we studied about selection of pH buffers.

### TEST EXAMPLE 4

### Stability Test Based on Difference of pH Buffer (6 Months)

(1) Stability of Liquid Preparation with Acetic Acid Buffer System

### [Method]

According to experimental formulations and target pH shown in Table 4 below, liquid preparations were prepared in the following manner.

Captisol (available from CyDex Inc.), sodium bisulfite (available from JUNSEI CHEMICAL), acetic acid (available from JUNSEI CHEMICAL), and sodium acetate (available from JUNSEI CHEMICAL) were added and dissolved in approximately 250 mL of purified water, to prepare a solution of inactive ingredients.

On the other hand, SUN N8075 was added and dissolved in approximately 50 mL of purified water, and the resultant solution was added to the above solution of inactive ingredients, and then the pH value was adjusted with 1 mol/L or 0.1 mol/L sodium hydroxide solution (available from Nacalai Tesque).

After filtering this solution under reduced pressure, 20 mL of this solution was filled into a brown ampule subjected to inner wall treatment by SiO₂ glass film (Silicoat ampule; brown 20 mL-volume; available from FUJI GLASS CO., LTD.), and then the ampule was sealed. Using high-pressure steam sterilizer (autoclave), the ampule was autoclaved in a setting condition: 121° C./24 minutes.

The samples were stored at 5° C., 25° C./60% relative humidity (RH), 45° C./75% RH and insoluble foreign matters were evaluated in time series until a lapse of 6 months, after evaluating presence/absence of insoluble foreign matters after autoclave sterilization. For evaluation of insoluble foreign matters, an appearance checker was used and observation was conducted under 1,000 Lux.

The pH value of the solution after 6-month storage at 45° C./75% RH was measured.

(2) Stability of Liquid Preparation with Amino Acid (Glycine) Buffer System

### [Method]

Likewise the foregoing (1) liquid preparation with acetic acid buffer system (for pH adjustment, 1 mol/L or 0.1 mol/L hydrochloric acid was used), each sample was prepared according to the experimental formulations and target pH shown in Table 4 below, and each sample was stored at 5° C., 25° C./60% relative humidity (RH), 45° C./75% RH and insoluble foreign matters were evaluated in time-series until a lapse of 6 months, after evaluating presence/absence of insoluble foreign matters after autoclave sterilization. Also the pH value of the solution after 6-month storage at 45° C./75% RH was measured.

TABLE 4

| | Formulations | | | | | | | |
|---|---|---|---|---|---|---|---|---|
| | 1 | 2 | 3 | 4 | 5 | 6 | 7 | 8 |
| | Buffer system | | | | | | | |
| | Acetic acid/sodium acetate | | | | Amino acid (glycine) | | | |
| | Buffer system | | | | Buffer system | | | |
| Captisol conc. (%) | 7.5 | 7.5 | 10.0 | 10.0 | 7.5 | 7.5 | 10.0 | 10.0 |
| SUN N8075 (g) | 2.5 | 2.5 | 2.5 | 2.5 | 2.5 | 2.5 | 2.5 | 2.5 |
| Captisol (g) | 37.5 | 37.5 | 50 | 50 | 37.5 | 37.5 | 50 | 50 |
| Sodium | 0.5 | 0.5 | 0.5 | 0.5 | 0.5 | 0.5 | 0.5 | 0.5 |
| bisulfite (g) | | | | | | | | |
| Glycine (g) | — | — | — | — | 3.75 | 3.75 | 3.75 | 3.75 |
| Acetic acid (100%) | 0.125 | 0.125 | 0.125 | 0.125 | — | — | — | — |
| (g) | | | | | | | | |
| Sodium acetate (g) | — | 0.095 | — | 0.095 | — | — | — | — |
| Hydrochloric acid | — | — | — | — | q.s. | q.s. | q.s. | q.s. |
| Sodium hydroxide | q.s. | q.s. | q.s. | q.s. | — | — | — | — |
| Adjusted pH | 3.8 | 4.2 | 3.8 | 4.2 | 3.8 | 4.2 | 3.8 | 4.2 |
| Purified water | q.s. | q.s. | q.s. | q.s. | q.s. | q.s. | q.s. | q.s. |
| Volume of | 500 | 500 | 500 | 500 | 500 | 500 | 500 | 500 |
| Preparation (mL) | | | | | | | | |

### [Results]

The results are collectively shown in Table 5 below.

In Table, ◯ means absence of insoluble matters, and × means presence of insoluble matters.

The content in parentheses represents a, storage period at which generation of insoluble matters was observed.

TABLE 5

| | | pH at preparation of | | |
|---|---|---|---|---|
| Captisol | Evaluation | solution | | |
| concentration | Item | pH: 3.8 | pH: 4.2 | Storage condition |

(1) Acetic acid/sodium acetate buffer system

| | | | | |
|---|---|---|---|---|
| 7.5% | Insoluble | ◯ | ◯ | Just after autoclave sterilization |
| (15 times amount)* | materials | ◯ | ◯ | 5° C./6 months |
| | | X | ◯ | 25° C./60% RH/6 months |
| | | (6 months) | | |
| | | ◯ | X | 40° C./60% RH/6 months |
| | | | (3 months) | |
| | pH | 2.74 | 2.96 | 40° C./75% RH/6 months |
| 10.0% | Insoluble | ◯ | ◯ | Just after autoclave |
| (20 times amount)* | materials | | | sterilization |
| | | ◯ | ◯ | 5° C./6 months |
| | | ◯ | ◯ | 25° C./60% RH/6 months |
| | | ◯ | ◯ | 40° C./75% RH/6 months |
| | pH | 2.60 | 2.82 | 40° C./75% RH/6 months |

(2) Amino acid (glycine) buffer system

| | | | | |
|---|---|---|---|---|
| 7.5% | Insoluble | ◯ | ◯ | Just after autoclave |
| (15 times amount)* | materials | | | sterilization |
| | | ◯ | ◯ | 5° C./6 months |
| | | ◯ | ◯ | 25° C./60% RH/6 months |
| | | ◯ | ◯ | 40° C./75% RH/6 months |
| | pH | 3.46 | 3.73 | 40° C./75% RH/6 months |
| 10.0% | Insoluble | ◯ | ◯ | Just after autoclave |
| (20 times amount)* | materials | | | sterilization |
| | | ◯ | ◯ | 5° C./6 months |
| | | ◯ | ◯ | 25° C./60% RH/6 months |
| | | ◯ | ◯ | 40° C./75% RH/6 months |
| | pH | 3.49 | 3.74 | 40° C./75% RH/6 months |

*weight ratio relative to active ingredient (SUN N8075)

From the above result, it can be understood that in preparation of the liquid preparation according to the present invention, using sodium bisulfite as an antioxidant, adding as a solubilizing agent Captisol in an amount of 5-15%, more preferably 7.5-13.5% (w/v), and adding as a pH buffer amino acid (glycine) buffer are advantageous in preventing generation of insoluble foreign matters and lowering of pH.

Secondly, it was found that a compound of the formula (I) or its salt represented by SUN N8075 which is an active ingredient of the present invention is decomposed by light in its aqueous solution so that a related substance which is indicative of oxidization and other related substances increase.

As a measure against light decomposition (including oxidization), it is necessary to take measures of, for example, adding an antioxidant, preferably sodium bisulfite, and filling into a brown glass container, however, these measures were sometimes insufficient in examination made by the present inventors.

As a result of examination made to clarify the cause, it was found that light decomposition is also ascribable to oxygen dissolved in the solution.

Then we examined for stability when oxygen dissolved in the solution was replaced by bubbling with inert gas such as nitrogen gas, argon gas, preferably with nitrogen gas.

### TEST EXAMPLE 5

### Examination on Prevention of Light Decomposition

(1) Examination on Prevention of Light Decomposition without Nitrogen Gas Replacement

### [Method]

To 800 mL of purified water, 100 g of Captisol (available from CyDex Inc.) serving as a solubilizing agent, 1 g of sodium bisulfite (available from JUNSEI CHEMICAL) serving as an antioxidant, and 5 g of SUN N8075 were sequentially dissolved, and the volume was adjusted to precisely 1,000 mL with purified water, and filtered under reduced pressure. Then each 20 mL of the solution was filled into a colorless ampule (Silicoat ampule, white, 20 mL capacity, available from FUJI GLASS CO., LTD.) and a brown ampule (Silicoat ampule, brown, 20 mL capacity, available from FUJI GLASS CO., LTD.), and then the ampule was sealed. Using autoclave (FLC-M09S30WZ, available from SAKURA SEIKI Co., Ltd.), autoclave sterilization was conducted in a setting condition: 121° C./24 minutes.

A sample of each of these ampules was stored under D65 lamp 2,000 Lux, and irradiated with 40, 80, 120×10⁴ Lux, and then SUN N8075 related substances were measured with a HPLC device. The related substances were calculated as a peak ratio of the total related substances, relative to SUN N8075 peak area.

(2) Examination on Prevention of Light Decomposition with Replacement by Nitrogen Gas Bubbling

### [Method]

To 150 mL of purified water, a predetermined amount (none, 0.05 g, 0.1 g and 0.2 g) of sodium bisulfite (available from JUNSEI CHEMICAL) serving as an antioxidant, and 1 g of SUN N8075 were sequentially dissolved, and the volume was adjusted to precisely 200 mL with purified water. After filtering under reduced pressure, the solution was bubbled with nitrogen gas for 5 minutes. Then 20 mL of the solution was immediately filled into a brown ampule (Silicoat ampule, brown, 20 mL capacity, available from FUJI GLASS CO., LTD.) having subjected to nitrogen gas replacement, and then the ampule was sealed. Using autoclave (FLC-M09S30WZ, available from SAKURA SEIKI Co., Ltd.), autoclave sterilization was conducted in a setting condition: 121° C./24 minutes.

A sample of each of these ampules was stored under D65 lamp 2,000 Lux, and irradiated with 40×10⁴ Lux, and then SUN N8075 related substances were measured with a HPLC device. The related substances were calculated as a peak ratio of the total related substances, relative to SUN N8075 peak area.

As a control sample, 1 g of SUN N8075 was dissolved into 150 mL of purified water, and the volume was adjusted precisely to 200 mL with purified water. Then the solution was filtered under reduced pressure, and bubbled with nitrogen gas for 5 minutes. Then 20 mL of the solution was immediately filled into a brown ampule (Silicoat ampule, brown, 20 mL capacity, available from FUJI GLASS CO., LTD.) replaced with nitrogen gas, and then the ampule was sealed.

This was prepared as a control sample at the start of the test, and SUN N8075 related substances were measured in the similar manner.

### [Results]

Examination results according to the above conditions (1) and (2) are collectively shown in Table 6 below.

TABLE 6

| | | | |
|---|---|---|---|
| | Irradiation | | Related |
| Solution preparation | condition | SUN N8075 | Substance |
| Condition/Color of ampule | (×10⁴ LUX) | Peak ratio | Peak ratio |

Control

| | | | |
|---|---|---|---|
| Without sodium bisulfite | Start | 99.37 | 0.629 |
| Brown ampule | | | |

(1) Without N₂ gas replacement

| | | | |
|---|---|---|---|
| Sodium bisulfite 0.1% | 40 | 98.86 | 1.140 |
| Captisol 10% | 80 | 98.70 | 1.301 |
| Brown ampule | 120 | 98.25 | 1.750 |
| Sodium bisulfite 0.1% | 40 | 95.45 | 4.552 |
| Captisol 10% | 80 | 95.05 | 4.953 |
| Colorless ampule | 120 | 94.01 | 5.987 |

(2) N₂ gas replacement

| | | | |
|---|---|---|---|
| Without sodium bisulfite | 40 | 99.03 | 0.967 |
| Brown ampule | | | |
| Sodium bisulfite 0.025% | 40 | 99.42 | 0.581 |
| Brown ampule | | | |
| Sodium bisulfite 0.05% | 40 | 99.39 | 0.608 |
| Brown ampule | | | |
| Sodium bisulfite 0.1% | 40 | 99.43 | 0.567 |
| Brown ampule | | | |

As can be seen from the above test examination results, it was found that adding an antioxidant, for example, sodium bisulfite, replacing dissolved oxygen in solution with nitrogen gas, and filling into a brown ampule enable prevention of light decomposition in the solution.

This point is clear in light of the fact that the brown ampule without nitrogen gas replacement failed to completely prevent light decomposition.

In the present invention, the antioxidant is preferably added in concentration of 0.02% or more, particularly preferably 0.05% or more, particularly in consideration of industrial production of liquid preparation employing nitrogen gas replacement.

From the foregoing examinations, a liquid preparation comprising a compound of the formula (I) represented by SUN N8075 or its pharmaceutically acceptable salt as an active ingredient provided by the present invention may be arranged to an objective liquid preparation by selecting a solubilizing agent for SUN N8075, particularly β-cyclodextrin derivative, selecting an antioxidant, for example, sulfite, bisulfite, pyrosulfite, α-thioglycerol and cysteine, selecting a pH adjuster, and selecting a pH buffer; adjusting the pH value of the solution within the range of from 3 to 5; and replacing dissolved oxygen in the pH-adjusted solution with nitrogen gas which is inert gas.

It was found that containing sodium bisulfite as an antioxidant is particularly preferred.

However, as described above, sodium bisulfite is likely to erode glass. Therefore, it may cause generation of insoluble matters (glass flakes) from glass during long-term storage.

According to examinations by the present inventors, the order of likelihood of formation of insoluble aggregate with free base of the compound represented by the formula (I) among various counter ions is estimated: sulfate ion>sulfite (or bisulfite ion)>>phosphate ion (or hydrogen phosphate ion, dihydrogen phosphate ion)>acetate ion>chloride ion>methane sulfonate ion.

Therefore, it is expected that addition of methane sulfonic acid, hydrochloric acid or sodium chloride which generate ions that are hard to form insoluble aggregates with free base of the compound represented by the formula (I) can prevent formation of insoluble aggregates.

Furthermore, since sulfate ion and sulfite ion (or bisulfite ion) are likely to form insoluble aggregates with free base of the compound represented by the formula (I), from the viewpoint as to preventing generation of insoluble aggregates caused by sulfite ion (or bisulfite ion) itself or sulfate, with taking the point that sulfate generates due to oxidation of sulfites or bisulfites into account, the adding amount of sodium bisulfite is preferably as small as possible, while it is particularly preferred as an antioxidant to be contained.

On the assumption that combined use of L-cysteine hydrochloride monohydrate which is proved to be a desirable antioxidant beside sodium bisulfite from the result of Test example 3, as an antioxidant to be contained will reduce the use amount of sodium bisulfite and prevent generation of sulfate due to oxidation of sulfites or bisulfites, which may prevent generation of insoluble foreign matters, we made a basic experiment (examination about adding ratio of L-cysteine hydrochloride monohydrate) for demonstrating this assumption.

These points were examined by the following tests.

### TEST EXAMPLE 6

### Examination on Two-Component Antioxidant

### (Examination on of Combined use of Sodium Bisulfite/L-Cysteine Hydrochloride Monohydrate)

### [Method]

According to the formulation of Tables 7 and 8, to 100-150 mL of purified water, sodium bisulfite (Japanese Pharmacopoeia; available from JUNSEI CHEMICAL), L-cysteine hydrochloride monohydrate (available from Nacalai Tesque), and SUN N8075 were added and dissolved in this order, and the volume was made precisely to 200 mL by addition of purified water.

After filtering under reduced pressure, the solution was bubbled with nitrogen gas for 5 minutes (not executed for the case “without nitrogen gas replacement”), and 20 mL of the solution was immediately filled into a brown ampule (Silicoat ampule, brown, 20 mL capacity, available from FUJI GLASS CO., LTD.) replaced with nitrogen gas (not executed for the case “without nitrogen gas replacement”), and then the ampule was sealed. Using autoclave (FLC-M09S30WZ, available from SAKURA SEIKI Co., Ltd.), autoclave sterilization was conducted in a setting condition: 121° C./24 minutes.

For each sample, insoluble foreign matters and odor were evaluated.

Evaluation of insoluble foreign matters was conducted using an appearance checker (under 1,000 Lux).

Also dissolved oxygen concentration in solution was measured.

### [Results]

The results are shown together in Tables 7 and 8.

TABLE 7

| | Concentration of | | | | | | |
|---|---|---|---|---|---|---|---|
| | added sodium bisulfite | | | | | | |
| | 0.025% | | | 0.05% | | | |
| SUN N8075 (g) | 1 | 1 | 1 | 1 | 1 | 1 | 1 |
| Sodium bisulfite (g) | 0.05 | 0.05 | 0.05 | 0.1 | 0.1 | 0.1 | 0.1 |
| L-cysteine HCl | — | 0.015 | 0.05 | — | 0.03 | 0.05 | 0.1 |
| monohydrate (g) | | | | | | | |
| Ratio thereof* | — | 3/10 | 1/1 | — | 3/10 | 1/2 | 1/1 |
| Purified water | q.s. | q.s. | q.s. | q.s. | q.s. | q.s. | q.s. |
| Total volume (mL) | 200 | 200 | 200 | 200 | 200 | 200 | 200 |
| N₂ gas replacement | done | Done | done | done | done | done | done |
| Dissolved oxygen | 0.52 | 1.88 | 0.82 | 0.82 | 1.85 | 0.91 | 0.88 |
| (ppm)/before | | | | | | | |
| sterilization | | | | | | | |
| Amount of insoluble | bit | Bit | great | bit | bit | great | great |
| matters/after | | | | | | | |
| sterilization | | | | | | | |
| Odor/ | — | — | Sulfur- | — | — | Sulfur- | Sulfur- |
| After sterilization | | | like | | | like | like |
| | | | odor | | | odor | odor |

*L-cysteine hydrochloride monohydrate/sodium bisulfite (w/w)

TABLE 8

| | Concentration of | | | | | | |
|---|---|---|---|---|---|---|---|
| | added sodium bisulfite | | | | | | |
| | 0.075% | | | 0.1% | | | |
| SUN N8075 (g) | 1 | 1 | 1 | 1 | 1 | 1 | 1 |
| Sodium bisulfite (g) | 0.15 | 0.15 | 0.15 | 0.15 | 0.2 | 0.2 | 0.2 |
| L-cysteine HCl | — | 0.05 | 0.1 | 0.5 | — | — | 0.1 |
| monohydrate (g) | | | | | | | |
| Ratio thereof* | — | 1/3 | 2/3 | 1/1 | — | — | 1/2 |
| Purified water | q.s. | q.s. | q.s. | q.s. | q.s. | q.s. | q.s. |
| Total volume (mL) | 200 | 200 | 200 | 200 | 200 | 200 | 200 |
| N₂ gas replacement | done | Done | done | done | Not | done | done |
| | | | | | done | | |
| Dissolved oxygen | 1.24 | 1.39 | 1.66 | 0.97 | 5.14 | 1.39 | 1.27 |
| (ppm)/before | | | | | | | |
| sterilization | | | | | | | |
| Amount of insoluble | bit | Bit | great | great | bit | bit | great |
| matters/after | | | | | | | |
| sterilization | | | | | | | |
| Odor/ | — | — | Sulfur- | Sulfur- | — | — | Sulfur |
| After sterilization | | | like | like | | | like |
| | | | odor | odor | | | odor |

*L-cysteine hydrochloride monohydrate/sodium bisulfite (w/w)

From the foregoing results, it was found that when the use amount of sodium bisulfite, which is an antioxidant against SUN N8075, is reduced, and L-cysteine hydrochloride monohydrate, which is also an antioxidant, is used in combination, the amount ratio, L-cysteine hydrochloride monohydrate/sodium bisulfite (w/w) is preferably not more than 1/3. When the amount of sodium bisulfite is varied with the amount ratio within the above range, quantity of insoluble matters formed is quite small, and no sulfur odor is observed as is the case with the use of only the sodium bisulfite aqueous solution. On the contrary, if the amount ratio is 1/2 or more, formation of large quantity of insoluble matters and strong sulfur odor are observed, which may be attributed to cystine that generates as a result of oxidation of L-cysteine, or to one or more kinds of substances generated by interaction between two or more kinds of substances selected from, or L-cysteine, cystine, or other degraded species of L-cysteine, SUN N8075, and sodium bisulfite.

From the above, it is expected that by adding L-cysteine hydrochloride monohydrate in an amount satisfying the above range of amount ratio, it is possible to reduce the adding amount of sodium bisulfite, prevent generation of insoluble aggregates, and thus develop liquid formulation having excellent effect of preventing the generation of glass flakes derived from glass container.

As a liquid preparation using an antioxidant selected from sulfite, bisulfite and pyrosulfite, and cysteine, Japanese Patent Laid-Open Publication No. Sho 63-132833 discloses an injectable preparation containing 3-methyl-1-phenyl-2-pyrazolon-5-on, and Edaravone injectable preparation using the compound, sodium bisulfite and L-cysteine hydrochloride monohydrate is put to practical use. However, Japanese Patent Laid-Open Publication No. Sho 63-132833 discloses that cysteine alone does not have stabilizing effect but combined use with an antioxidant will exert stabilizing activity, for the problem that the compound is easily oxidized by dissolved oxygen and generates insoluble foreign matters. In the publication, it is disclosed that, as a ratio of the use amounts of two components, the antioxidant is 0.001-0.5 w/v % (particularly, 0.01-0.2 w/v % is preferred), and cysteine is 0.005-0.5 w/v % (particularly, 0.01-0.2 w/v % is preferred), and as examples, those having L-cysteine hydrochloride/sodium pyrosulfite (w/w) of 1/2, and L-cysteine hydrochloride/sodium bisulfite (w/w) of 1/2. Also in Edaravone injectable preparation, L-cysteine hydrochloride/sodium bisulfite (w/w) is 1/2.

On the other hand, in the present invention, L-cysteine hydrochloride monohydrate alone has anti-oxidation effect for the active ingredient in the present invention as shown in Test example 3. In the case of an active ingredient of the present invention, it is necessary to restrain not only chemical change such as oxidization but also generation of insoluble matters caused by physiochemical property of the active ingredient. In addition to this, two kinds of antioxidant substances were used in combination in light of restraint of formation of insoluble matters derived from glass container. In other words, the former measure is based on the point that the active ingredient and sulfites or bisulfites or sulfate are presumed to be very likely to form insoluble aggregates and the point that sulfate is generated by self-oxidation of sulfites or bisulfites, and the latter measure is based on the assumption that insoluble matters (glass flakes) are likely to generate from glass container by erosion of inner wall of glass container by sulfites or bisulfites or sulfate. By adding L-cysteine hydrochloride monohydrate with a weight ratio of 1/10-1/3 relative to sulfite, preferably to sodium bisulfite, it is possible to reduce sulfites or bisulfites which are likely to form insoluble aggregates with the active ingredient while keeping anti-oxidation activity for the active ingredient; and to prevent sulfites or bisulfites from being oxidized to sulfate. Accordingly, the effect of preventing generation of insoluble aggregates by the active-ingredient and sulfites or bisulfites and sulfate, while preventing erosion of glass container, can be expected.

The compound of formula (I) represented by SUN N8075 or its pharmaceutically acceptable salt, which is an active ingredient in the liquid preparation provided by the present invention, has high surface activity, so that the solution dissolving the same is likely to generate insoluble foreign matters due to surface activity (interfacial adsorption and self aggregation) when contacting interface increases. Furthermore, high foaming property and low defoaming property in the solution state will bring a problem in industrial scale production, and tend to cause difficulties in administration of the liquid preparation. Furthermore, high absorptivity may cause filter absorption in sterile filtration and cause clogging of filter.

Therefore, in practical production, it is necessary to reduce the surface activity of a solution containing such an active ingredient in order to avoid these problems.

In view of the above, we examined the effect of reducing surface activity of a solution containing an active ingredient by adding a substance, which is expected to restrain the surface activity coming from the active ingredient.

### TEST EXAMPLE 7

### Examination on Reduction in Surface Activity in SUN N8075 Aqueous Solution

### [Method]

Substances shown in Table 10 below were used. Into each test compound solution which was prepared in advance to achieve each adding concentration shown in the Table, SUN N8075 was added and dissolved so that the concentration thereof was 5 mg/mL, and the pH value was adjusted to 3 to 4 as appropriate by hydrochloric acid to prepare a sample solution.

Each 10 mL of sample solution was put into a 10-mL stoppered test tube, and shaken 30 strokes vertically by hand and allowed to stand upright, and under room diffused light, appearance of sample solution after shaking, foaming property (foaming quantity), defoaming time, post standing observation (after standing for 3 days or more) were evaluated.

As a control, an aqueous solution containing no test substance but containing 5 mg/mL of SUN N8075 was examined in a similar manner.

### [Results]

Evaluation in each test item, and marks used in general judgment are shown in Table 9 below.

The results are collectively shown in Table 10.

TABLE 9
Definition of mark used in judgment
Appearance after shaking/Appearance after standing

| | |
|---|---|
| A | Clear |
| B+ | Almost clear |
| B | Much less insoluble matters than Control |
| C | Clearly less insoluble matter than Control |
| D | Comparable amount of insoluble matters with Control |
| E | More insoluble matters than Control |

Large amount of insoluble matters in wall face and appearance in Control

Foaming property

| | |
|---|---|
| A | Much less foaming than Control |
| B | Clearly less foaming than Control |
| C | Slightly less foaming than Control |
| D | Comparable to Control |
| E | More foaming than Control |

Very high foaming property in Control

Defoaming time

| | |
|---|---|
| A | In 20 minutes |
| B | In 60 minutes |
| C | 60 minutes to 3 days |
| D | Comparable to Control |

Not defoamed after standing for 1 week in Control

General judgment

| | |
|---|---|
| A | Great effect |
| B+ | Large effect |
| B | Effective |
| C | Effective but very small |
| D | Comparable to Control (No effect) |
| E | Inferior to Control (clouded) |

TABLE 10

| | | Appearance | | | Appearance | |
|---|---|---|---|---|---|---|
| | | after | Foaming | Defoaming | after | General |
| Ingredient/concentration | | shaking | property | time | standing | judgment |
| Ethanol | 1.00% | B | C | C | B | B |
| | 2.50% | B | D | — | C | C |
| | 5.00% | D | D | — | E | D |
| | 10.00% | D | D | — | E | D |
| Propylene | 1.00% | B | B | C | A | B+ |
| glycol | 2.50% | C | D | — | D | D |
| | 5.00% | B | D | — | D | D |
| | 10.00% | C | D | — | D | D |
| Chlorobutanol | 0.10% | D | C | D | D | D |
| | 0.25% | C | C | C | A | B |
| | 0.50% | B | B | C | A | B+ |
| Captisol | 10.00% | A | A | A | A | A |
| Sodium chloride | 25 mM | D | B | D | D | D |
| | 50 mM | B | A | C | B | C |
| | 75 mM | A | B+ | A | A | A |
| | 100 mM | A | B | A | A | A |
| Meglumine | 25 mM | C | D | C | C | C |
| | 50 mM | A | C | B | A | B |
| | 75 mM | A | B | B | A | A |
| | 100 mM | A | C | A | A | A |
| Methane | 15 mM | D | B | C | D | D |
| sulfonic acid | | | | | | |
| Glycine | 100 mM | B | B | C | B | B |
| Sodium L- | 30 mM | B+ | B+ | C | B+ | B+ |
| glutamate | 60 mM | A | B+ | A | A | A |
| L-Arginine | 30 mM | B+ | B | B | B+ | B+ |
| | 60 mM | A | A | A | A | A |
| L-Histidine | 60 mM | A | A | A | A | A |

As can be seen from these results, in the liquid preparation comprising a compound of formula (I) and its pharmaceutically acceptable salt provided by the present invention, by adding at least one selected from the group consisting of sodium chloride, meglumine, L-arginine, L-glutamate, propylene glycol, ethanol and chlorobutanol as a substance for reducing surface activity, it is possible to reduce the surface activity of the solution preparation, improve the productivity of liquid preparation, and reduce demerit in administration.

In addition, from the above examination, it can be considered that β-cyclodextrin derivative (representative example: Captisol) serving as a solubilizing agent for SUN N8075, and glycine or L-histidine which forms an amino acid buffer system serving as a pH buffer reduce the surface activity of the solution, and use of these additives is particularly preferred also in that point. As a buffer, glycine is particularly preferred.

From the Test example described above, the basic form of the liquid preparation comprising a compound of formula (I) represented by SUN N8075 and its pharmaceutically acceptable salt provided by the present invention is a liquid preparation which comprises:

(a) at least one selected from sulfite, bisulfite, pyrosulfite, α-thioglycerol and cysteine,

(b) a β-cyclodextrin derivative, and

(c) a pH buffer,

wherein the pH value is adjusted to fall within a range of from 3 to 5.

In addition to this basic form, dissolved oxygen may be reduced, preferably to 4 ppm or less, by inert gas replacement, and a substance that reduces surface activity derived from the active ingredient, preferably, at least one selected from the group consisting of sodium chloride, meglumine, L-arginine, glycine, L-histidine, L-glutamate, β-cyclodextrin derivative, propylene glycol, ethanol and chlorobutanol may be included. Furthermore, a substance that prevents generation of insoluble aggregates with free base of the compound of formula (I), preferably, at least one selected from the group consisting of methane sulfonic acid, hydrochloric acid and sodium chloride may also be included. As a result, a clinically applicable liquid preparation is realized.

Examination made by the present inventors revealed that such liquid preparation can be desirably prepared in the following steps.

Concretely, it is a method of producing a liquid preparation comprising the steps of:

(a) preparing a solution of drug substance by dissolving a compound of the formula (I) represented by SUN N8075 or its pharmaceutically acceptable salt in water in which dissolved oxygen is reduced by inert gas replacement;

(b) preparing a solution of inactive ingredients by dissolving in water, a solubilizing agent such as β-cyclodextrin derivative, a pH buffer and a pH adjuster, adjusting the pH value to 3-5, replacing dissolved oxygen by inert gas replacement, and dissolving at least one selected from the group consisting of sulfite, bisulfite, pyrosulfite, α-thioglycerol and cysteine; and

(c) adding and mixing the solution of drug substance to the solution of inactive ingredients under inert gas flow.

Examination results concerning actual formulation tests, and long-term stability of the liquid preparation obtained therefrom according to such a production method will be given below. Since the present liquid preparation is directly administered by injection or drip, it is required that insoluble foreign matters will not be generated during storage. Therefore, in evaluations of the following tests, observation on generation of insoluble matters was used as an index.

### Formulation Test 1: Examination of Basic Formulation

### [Basic Process: Formulation No. 1]

Approximately 2 L of purified water was taken in a stainless beaker, and 277.5 g of Captisol (available from Clinical Grade; CyDex Inc.), and 27.75 g of glycine (guaranteed grade; available from Nacalai Tesque) were sequentially added under stirring by a magnetic stirrer. This solution was aerated with nitrogen gas in an aerating condition appropriate to reduce dissolved oxygen to 2 ppm or less.

On the other hand, 18.5 g of SUN N8075 and approximately 500 mL of purified water in which dissolved oxygen had been reduced to 2 ppm or less by nitrogen gas aeration were added to a glass beaker, and SUN N8075 was dissolved by stirring by a magnetic stirrer and ultrasonic irradiation to prepare a solution of drug substance.

Under nitrogen gas flow, 3.7 g of sodium bisulfite (Japanese Pharmacopoeia; available from JUNSEI CHEMICAL) was added and dissolved into the solution in the stainless beaker, and the solution of drug substance was gently added. Then, the pH value thereof was adjusted to about 4.0 with the use of a pH meter (NAVI hF-52, HORIBA Co., Ltd.) by adding 21.8 g of 1 mol/L hydrochloric acid (for volumetric analysis, available from Nacalai Tesque), and purified water in which dissolved oxygen had been reduced to 2 ppm or less by nitrogen gas replacement was added to make the total volume 3,700 mL, and then mixture was stirred thoroughly with a magnetic stirrer.

The solution was filtered through a hydrophilic PVDF filter having a pore size of 0.22 μm (Durapore 0.22 μm GVφ45, available from Millipore) under pressure of nitrogen, and received in a reserve tank under nitrogen flow. With an automatic ampule filling/sealing machine (NF-2, available from ASAHI SEIKI) and a brown ampule having an inner wall treated with SiO₂ glass film (Silicoat ampule, brown, 20 mL capacity, available from FUJI GLASS CO., LTD.), the interior of the ampule was replaced by nitrogen and 20 mL of the solution was filled and the ampule was immediately sealed.

It was confirmed that dissolved oxygen in the content in the sealed ampule was 2 ppm or less.

Using a high-pressure steam sterilizer (FLC-M09S30WZ, available from SAKURA SEIKI Co., Ltd.), autoclave sterilization was conducted in a setting condition: 121° C./24 minutes.

According to the basic process as described above, liquid preparations of Formulations No. 1 to 7 were produced according to the formulation shown in Table 11 (Preparation scales vary according to the formulation, 2800-5500 mL).

For each liquid preparation of the respective Formulation No., whether or not insoluble foreign matters are generated in the storage condition was evaluated.

### [Evaluation Method]

### Insoluble Foreign Matters

Samples were stored in respective conditions of 40° C./75% RH, 25° C./60% RH, and 60° C., and insoluble foreign matters in samples were evaluated in time-series using an appearance checker (observed under 1,000 Lux).

### [Results]

These results are collectively shown in Table 11.

Marks used in this Table represent the following meanings.
o: No insoluble matter generated
nM: Insoluble matter generated in n-month storage

TABLE 11

| | | Formulation No. | | | | | | |
|---|---|---|---|---|---|---|---|---|
| | | 1 | 2 | 3 | 4 | 5 | 6 | 7 |
| SUN N8075 (mg/mL) | | 5 | 5 | 5 | 5 | 5 | 5 | 5 |
| Captisol (%) | | 7.5 | 10 | 10 | 10 | 10 | 10 | 10 |
| Glycine (mM) | | 100 | 100 | 100 | 100 | 100 | 100 | 100 |
| Sodium bisulfite (%) | | 0.1 | 0.1 | 0.1 | 0.1 | 0.05 | 0.05 | 0.1 |
| L-cysteine hydrochloride | | — | — | — | — | — | 0.015 | — |
| monohydrate (%) | | | | | | | | |
| Methane sulfonic acid (mM) | | — | — | — | — | — | — | 15 |
| Hydrochloric acid* | | q.s. | q.s. | q.s. | q.s. | q.s. | q.s. | q.s. |
| Sodium hydroxide* | | q.s. | q.s. | q.s. | q.s. | q.s. | q.s. | q.s. |
| Purified water | | q.s. | q.s. | q.s. | q.s. | q.s. | q.s. | q.s. |
| pH | | 4.0 | 3.8 | 4.0 | 4.2 | 4.0 | 4.0-4.3 | 4.0 |
| (targeted at preparation) | | | | | | | | |
| Filling amount (mL) | | 20 | 20 | 20 | 20 | 20 | 20 | 20 |
| Filling container | | Brown | Brown | Brown | Brown | Brown | Brown | Brown |
| (color of ampule) | | | | | | | | |
| N₂ gas replacement | | done | done | done | done | done | done | done |
| (dissolved oxygen ≦2 ppm) | | | | | | | | |
| Autoclave sterilization | | done | done | done | done | done | done | done |
| (121° C./24 minutes) | | | | | | | | |
| Storage | Storage | | | | | | | |
| condition | period | | | | | | | |
| 40° C./75% RH | 6 months | 1 M | 1 M | 1 M | 3 M | ◯ | ◯ | ◯ |
| 25° C./60% RH | 24 months | 1 M | 1 M | 1 M | 1 M | ◯ | ◯ | ◯ |
| 60° C. | 3 months | 1 M | 1 M | 1 M | 3 M | 2 M | ◯ | ◯ |

*added as required

From the examination of basic formulation, it was demonstrated that Formulations 6 and 7 were excellent in insoluble matter preventing ability. When autoclave sterilization was not conducted in the same formulation as Formulation 6, insoluble foreign matters were not also occurred in the same storage condition.

Formulation 6 uses a two-component antioxidant in which sodium bisulfite and L-cysteine hydrochloride monohydrate are used as antioxidants in combination. Formulation 7 is a formulation in which methane sulfonic acid, being a substance that prevents generation of insoluble aggregates with free base of compound of formula (I) is added.

Specific formulations in which other additive substances (surface activity suppressing substance) are added to this formulation were examined. In the following tests, the process is based on a method where autoclave sterilization is not conducted and hence heat history is mild, in consideration of commercial production, taking assurance of stability in the market distribution.

### Formulation Test 2: Examination of Specific Formulation (Part 1)

### [Basic Process: Formulation No. 8]

Approximately 2 L of purified water was taken in a stainless beaker, and 300 g of Captisol (Clinical Grade; available from CyDex Inc.), and 22.5 g of glycine (guaranteed grade; available from Nacalai Tesque) were sequentially added under stirring by a magnetic stirrer. After adding 4.32 g of methane sulfonic acid (guaranteed grade; available from Wako Pure Chemical Industries Co., Ltd.), the pH value was adjusted to about 4.2, measured by a pH meter (NAVI hF-52, HORIBA Co., Ltd.), by adding 1 mol/L sodium hydroxide (for volumetric analysis, available from Nacalai Tesque). This solution was aerated with nitrogen gas in an aerating condition appropriate to reduce dissolved oxygen to 2 ppm or less.

On the other hand, 15 g of SUN N8075 and approximately 300 mL of purified water in which dissolved oxygen had been reduced to 2 ppm or less by nitrogen gas aeration were added to a glass beaker, and SUN N8075 was dissolved by stirring by a magnetic stirrer and ultrasonic irradiation to prepare an solution of drug substance.

Under nitrogen gas flow, 31.14 g of propylene glycol (guaranteed grade; available from Nacalai Tesque) was added and 1.5 g of sodium bisulfite (Japanese Pharmacopoeia; available from JUNSEI CHEMICAL) was added and dissolved into the solution in the stainless beaker, and then the solution of drug substance was gently added. After adjusting the total volume to 3,000 mL by adding 1 mmol/L methane sulfonic acid in which dissolved oxygen had been reduced to 2 ppm or less by nitrogen gas replacement, the mixture was stirred thoroughly with a magnetic stirrer.

The solution was filtered through hydrophilic PVDF filters having pore sizes of 0.45 μm and 0.22 μm (Milipack 60 (0.45 μm and 0.22 μm, respectively), available from Millipore) under pressure of nitrogen, and received in a reserve tank under nitrogen flow. With an automatic ampule filling/sealing machine (NF-2, available from ASAHI SEIKI) and a brown ampule having an inner wall treated with SiO₂ glass film (Silicoat ampule, brown, 20 mL capacity, available from FUJI GLASS CO., LTD.), the interior of the ampule was replaced by nitrogen and 20 mL of the solution was filled and immediately sealed.

It was confirmed that the dissolved oxygen in the content in the sealed ampule was 2 ppm or less.

According to the basic process as described above, liquid preparations of Formulations No. 8 to 16 were produced according to precipitation shown in Table 12. (Preparation scale is 3000 mL for all formulations).

For each liquid preparation of the respective Formulation No., whether or not insoluble foreign matters are generated in the storage condition was evaluated.

### [Evaluation Method]

### Insoluble Foreign Matters

### 1. Temperature/Humidity Conditions

Samples were stored in respective conditions of 40° C./75% RH, 25° C./60% RH, and 60° C., and insoluble foreign matters in samples were evaluated in time-series using an appearance checker (observed under 1,000 Lux).

### [Results]

These results are collectively shown in Table 12.

Marks used in this Table represent the following meanings.
o: No insoluble matter generated
nM: insoluble matter generated in n-month storage

TABLE 12

| | | Formulation No. | | | | | | | | |
|---|---|---|---|---|---|---|---|---|---|---|
| | | 8 | 9 | 10 | 11 | 12 | 13 | 14 | 15 | 16 |
| SUN N8075 (mg/mL) | | 5 | 5 | 5 | 5 | 5 | 5 | 5 | 5 | 5 |
| Captisol (%) | | 10 | 10 | 10 | 10 | 10 | 10 | 10 | 10 | 10 |
| Glycine (mM) | | 100 | 100 | 50 | 100 | 50 | 50 | 100 | 100 | — |
| Sodium bisulfite (%) | | 0.05 | 0.05 | 0.05 | 0.05 | 0.05 | 0.05 | 0.05 | 0.05 | 0.05 |
| L-cysteine hydrochloride | | — | 0.015 | 0.015 | 0.015 | 0.015 | 0.015 | 0.015 | 0.015 | 0.015 |
| Monohydrate (%) | | | | | | | | | | |
| Methane sulfonic acid (mM) | | 15 | 15 | 15 | 15 | 15 | 15 | — | 15 | 15 |
| L-histidine (mM) | | — | — | — | — | — | — | — | — | 30 |
| Sodium chloride (mM) | | — | — | — | — | — | — | 15 | — | — |
| Meglumine (mM) | | — | — | — | — | — | 75 | — | — | — |
| Propyleneglycol (% v/v) | | 1.0 | 1.0 | 1.0 | 1.5 | 0.5 | 1.0 | 1.0 | — | 1.0 |
| Ethanol (% v/v) | | — | — | — | — | — | — | — | 1.0 | — |
| Hydrochloric acid* | | q.s. | q.s. | q.s. | q.s. | q.s. | q.s. | q.s. | q.s. | q.s. |
| Sodium hydroxide* | | q.s. | q.s. | q.s. | q.s. | q.s. | q.s. | q.s. | q.s. | q.s. |
| Purified water | | q.s. | q.s. | q.s. | q.s. | q.s. | q.s. | q.s. | q.s. | q.s. |
| pH | | 4.0 | 3.8 | 3.7-3.8 | 3.6-4.0 | 3.6-4.0 | 3.7-3.8 | 3.7-3.8 | 3.7-3.8 | 3.7-3.8 |
| (targeted at preparation) | | | | | | | | | | |
| Filling amount (mL) | | 20 | 20 | 20 | 20 | 20 | 20 | 20 | 20 | 20 |
| Filling container | | Brown | Brown | Brown | Brown | Brown | Brown | Brown | Brown | Brown |
| (color of ampule) | | | | | | | | | | |
| N₂ gas replacement | | done | done | done | done | done | done | done | done | done |
| (dissolved oxygen ≦2 ppm) | | | | | | | | | | |
| Autoclave sterilization | | Not | Not | Not | Not | Not | Not | Not | Not | Not |
| (121° C./24 minutes) | | done | done | done | done | done | done | done | done | done |
| Storage | Storage | | | | | | | | | |
| condition | period | Evaluation of generation of insoluble matters | | | | | | | | |
| 40° C./75% RH | 6 months | ◯ | ◯ | ◯ | ◯ | ◯ | ◯ | ◯ | ◯ | ◯ |
| 25° C./60% RH | 18 months | 9 M | ◯ | ◯ | ◯ | ◯ | ◯ | ◯ | ◯ | ◯ |
| 60° C. | 3 Months | 1 M | ◯ | ◯ | ◯ | ◯ | ◯ | ◯ | ◯ | ◯ |

*added as required

As is evidenced from the result shown in Table 12, excellent insoluble matter preventing effect is achieved by adding methane sulfonic acid and/or a surface activity suppressing substance to the base of the formulation of Formulation No. 6.

Addition of L-cysteine hydrochloride monohydrate is necessary for preventing insoluble matters, particularly in the storage condition of 60° C.

### Formulation Test 3: Examination of Specific Formulation (Part 2)

In consideration of commercial distribution, generation of insoluble matter in various stressed conditions in addition to long-term storage condition and accelerated condition was evaluated.

### [Basic Process: Formulation No. 17]

Approximately 2 L of purified water was taken in a stainless beaker, and 420 g of Captisol (available from Clinical Grade; CyDex Inc.), and 31.5 g of glycine (guaranteed grade; available from Nacalai Tesque) were sequentially added into the beaker and dissolved under stirring with a magnetic stirrer. By adding 1 mol/L hydrochloric acid (for volumetric analysis, available from Nacalai Tesque), the pH value was adjusted to about 4.0, measured by a pH meter (NAVI hF-52, HORIBA Co., Ltd.). This solution was aerated with nitrogen gas in an aerating condition appropriate to reduce dissolved oxygen to 2 ppm or less.

21 g of SUN N8075 and approximately 300 mL of purified water in which dissolved oxygen had been reduced to 2 ppm or less by nitrogen gas aeration were added to a glass beaker, and SUN N8075 was dissolved by stirring by a magnetic stirrer and ultrasonic irradiation to prepare a solution of drug substance.

Under nitrogen gas flow, 2.1 g of sodium bisulfite (Japanese Pharmacopoeia; available from JUNSEI CHEMICAL) and 0.63 g of L-cysteine hydrochloride monohydrate (guaranteed grade; available from Nacalai Tesque) were sequentially and immediately added and dissolved into the solution in the stainless beaker, and then the solution of drug substance was gently added. After adjusting the total volume to 4,200 mL by adding 1 mmol/L hydrochloric acid in which dissolved oxygen had been reduced to 2 ppm or less by nitrogen gas replacement, the mixture was stirred thoroughly with a magnetic stirrer.

The solution was filtered through a hydrophilic PVDF filter having a pore size of 0.22 μm (Milipack 60 (0.22 μm), available from Millipore) under pressure of nitrogen, and received in a reserve tank under nitrogen flow. With an automatic ampule filling/sealing machine (NF-2, available from ASAHI SEIKI) and a brown ampule having inner wall treated with SiO₂ glass film (Silicoat ampule, brown, 20 mL capacity, available from FUJI GLASS CO., LTD.), the interior of the ampule was replaced by nitrogen and 20 mL of the solution was filled and immediately sealed.

It was confirmed that the dissolved oxygen in the content in the sealed ampule was 2 ppm or less.

According to the basic process as described above, liquid preparations of Formulations-No. 17 to 27 were produced according to precipitation shown in Table 13. (Preparation scales vary according to the formulation, 3800-5800 mL)

In Formulation No. 27, a brown vial having an inner wall treated with SiO₂ glass film (Silicoat vial, brown, 30 mL capacity, available from FUJI GLASS CO., LTD.) was filled with 29-30 mL, and stoppered and sealed with a Teflon (registered name) coated butyl rubber stopper(available from Daikyo Seiko Co., Ltd.) and an aluminum/plastic flip-off cap (Flip cap 20; available from Hisa Kinzoku Kogyo Co., Ltd.).

For each liquid preparation of the respective Formulation No., whether or not insoluble foreign matters were generated in the storage condition was evaluated.

### [Evaluation Method]

### Insoluble Foreign Matters

### 1. Accelerated Condition

Each test sample was stored in a condition of 40° C./75% RH (humidity), and insoluble foreign matters were evaluated in time-series (observed under 1,000 Lux).

### 2. Long-Term Storage Condition

Each test sample was stored in a condition of 25° C./60% RH (humidity), and insoluble foreign matters were evaluated in time-series (observed under 1,000 Lux).

### 3. Stressed Condition

### (3.1) High-Temperature Condition

Each test sample was stored at 60° C, and insoluble foreign matters were evaluated in time-series (observed under 1,000 Lux).

### (3.2) Low-Temperature Condition

Each test sample was stored at 5° C., and insoluble foreign matters were evaluated in time-series (observed under 1,000 Lux).

### (3.3) Light Exposure Condition

Each test sample was stored under 2,000 Lux by a D65 lamp, and insoluble foreign matters were evaluated in time-series (observed under 1,000 Lux).

### [Results]

These results are collectively shown in Table 13.

Marks used in this Table represent the following meanings.
o: No insoluble matter generated
nM: Insoluble matters generated in n-month storage
nW: Insoluble matters observed at n-week storage
25 days: Insoluble matters observed at end of 25-day test period

TABLE 13

| | Formulation No. | | | | | | | | | | |
|---|---|---|---|---|---|---|---|---|---|---|---|
| | 17 | 18 | 19 | 20 | 21 | 22 | 23 | 24 | 25 | 26 | 27 |
| SUN N8075 (mg/mL) | 5 | 5 | 5 | 5 | 5 | 5 | 5 | 5 | 5 | 5 | 5 |
| Captisol (%) | 10 | 10 | 10 | 10 | 10 | 10 | 10 | 10 | 10 | 10 | 10 |
| Glycine (mM) | 100 | 100 | 100 | 100 | 100 | 100 | 100 | 100 | 100 | 100 | 100 |
| Sodium bisulfite (%) | 0.05 | 0.05 | 0.05 | 0.05 | 0.1 | 0.05 | 0.05 | 0.05 | 0.05 | 0.05 | 0.05 |
| L-cysteine hydrochloride | 0.015 | 0.015 | 0.015 | 0.015 | 0.03 | 0.015 | 0.015 | 0.015 | 0.015 | 0.015 | 0.015 |
| Monohydrate (%) | | | | | | | | | | | |
| Methane sulfonic acid (mM) | — | — | — | — | — | 15 | — | — | — | — | — |
| Sodium chloride (mM) | — | — | — | — | — | — | 15 | 15 | — | 15 | — |
| Propyleneglycol (% v/v) | — | — | — | — | — | — | — | — | 1.0 | 1.0 | — |
| Hydrochloric acid* | q.s. | q.s. | q.s. | q.s. | q.s. | q.s. | q.s. | q.s. | q.s. | q.s. | q.s. |
| Sodium hydroxide* | q.s. | q.s. | q.s. | q.s. | q.s. | q.s. | q.s. | q.s. | q.s. | q.s. | q.s. |
| Purified water | q.s. | q.s. | q.s. | q.s. | q.s. | q.s. | q.s. | q.s. | q.s. | q.s. | q.s. |
| pH | 3.7-4.0 | 3.1-3.5 | 4.3-4.6 | 3.7-4.0 | 3.7-4.0 | 3.7-4.0 | 3.1-3.5 | 3.7-4.0 | 3.7-4.0 | 3.7-4.0 | 3.7-4.0 |
| (targeted at preparation) | | | | | | | | | | | |

Evaluation of generation of insoluble matters
<Accelerated condition, Long-term storage condition>

| | | | | | | | | | | | | |
|---|---|---|---|---|---|---|---|---|---|---|---|---|
| 40° C./75% RH | 6 M | ◯ | ◯ | ◯ | ◯ | ◯ | ◯ | 3 M | ◯ | ◯ | ◯ | ◯ |
| 25° C./60% RH | 12 M | ◯ | ◯ | ◯ | ◯ | ◯ | ◯ | 6 M | ◯ | ◯ | ◯ | ◯ |

<Stressed condition>

| | | | | | | | | | | | | |
|---|---|---|---|---|---|---|---|---|---|---|---|---|
| 5° C. | 12 M | ◯ | ◯ | ◯ | ◯ | ◯ | ◯ | 12 M | ◯ | ◯ | ◯ | ◯ |
| 60° C. | 12 W | ◯ | ◯ | ◯ | ◯ | ◯ | ◯ | 4 W | ◯ | ◯ | ◯ | ◯ |
| 2,000 Lux/hr | 25 days | ◯ | ◯ | ◯ | ◯ | ◯ | ◯ | 25 days | ◯ | ◯ | ◯ | ◯ |
| (D65 Type Lump) | (120 × 10⁴ | | | | | | | | | | | |
| | Lux) | | | | | | | | | | | |

*added as required

This formulation test example evaluates stability (generation of insoluble matters) in accelerated condition, long-term storage condition and stressed condition of the liquid preparation according to the present invention.

As is evidenced by the results shown in Table 13, it can be understood that the liquid preparation comprising a compound of the formula (I) represented by SUN N8075 or its pharmaceutically acceptable salt provided by the present invention is excellent in stability, and generation of insoluble matters is not observed even in a long-term storage. Furthermore, it can be understood that various substances that reduce surface activity coming from an active ingredient, and substances that prevent generation of insoluble aggregates with free base of the compound of formula (I) could be added singly or in combination. In the case of adding sodium chloride, it is necessary that the pH value should be adjusted to 3.8 or higher.

As described above, according to the present invention, for a compound represented by the formula (I) and its pharmaceutically acceptable salt having very low solubility at pH around neutrality, selection of a solubilizing agent, particularly β-cyclodextrin derivative, selection of an antioxidant, e.g., sulfite, bisulfite, pyrosulfite, α-thioglycerol and cysteine, selection of a pH adjuster, selection of a pH buffer, and adjustment of the pH value of solution within the range of from 3 to 5, and furthermore, replacement of dissolved oxygen by nitrogen gas which is an inert gas, addition of a substances capable of reducing surface activity or a substance capable of preventing generation of insoluble aggregates with free base of the compound of formula (I) as appropriate in preparing a solution enable preparation of an objective liquid preparation.

Such a liquid preparation stably contains the dissolved compound represented by the formula (I) and its pharmaceutically acceptable salt and has excellent long-term stability.

Therefore, the most preferred liquid preparation provided by the present invention is a liquid preparation containing 1 to 5 mg/mL of (2S)-1-(4-amino-2,3,5-trimethylphenoxy)-3-{4-[4-(4-fluorobenzyl)phenyl]-1-piperazinyl}-2-propanol or its pharmaceutically acceptable salt, 0.05 to 0.10% (w/v) of sodium bisulfite, 0.015 to 0.03% (w/v) of L-cysteine hydrochloride monohydrate, 7.5 to 10% (w/v) of β-cyclodextrin sulfobutyl ether sodium salt, 50 to 100 mM of glycine, and hydrochloric acid, wherein the pH value thereof falls within a range of from 3.0 to 4.5.

A liquid preparation provided by the present invention may be directly administered by injection or by drip, and may be used in therapy of a target disease by appropriately adjusting the dose.

### INDUSTRIAL APPLICABILITY

The present invention provides a stable liquid preparation which comprises, as an active ingredient, the above compound represented by the formula (I) having very poor solubility at pH around neutrality and its pharmaceutically acceptable salt.

A liquid preparation provided by the present invention stably contains a compound represented by the formula (I) and its pharmaceutically acceptable salt which is an active ingredient, and since such compound and its pharmaceutically acceptable salt have excellent remedial and therapeutic ability against symptoms based on ischemic disorder and neurodegenerative disease, symptoms from spasm, epilepsy and migraine, as well as various symptoms caused by diabetes, arteriosclerosis and inflammatory disease, the liquid preparation is highly effective for therapy of these diseases, and thus provides great industrial applicability.

1. A liquid preparation comprising, as an active ingredient, (2S)-1-(4-amino-2,3,5-trimethylphenoxy)-3-{4-[4-(4-fluorobenzyl)-phenyl]-1-piperazinyl}-2-propanol represented by the formula (I) or its pharmaceutically acceptable salt, the preparation further comprising:
(a) at least one selected from the group consisting of sulfite, bisulfite, pyrosulfite, α-thioglycerol and cysteine;
(b) a β-cyclodextrin derivative; and
(c) a pH buffer,
wherein a pH value thereof is adjusted to fall within a range of from 3 to 5.

2. The liquid preparation according to claim 1, comprising (a) at least one selected from the group consisting of sodium bisulfite, sodium pyrosulfite, α-thioglycerol, L-cysteine hydrochloride monohydrate and L-cysteine.

3. The liquid preparation according to claim 1, comprising (a) sodium bisulfite.

4. The liquid preparation according to claim 1, comprising (a) sodium bisulfite and L-cysteine hydrochloride monohydrate.

5. The liquid preparation according to claim 4, wherein a quantity ratio of sodium bisulfite and L-cysteine hydrochloride monohydrate is between 1/10 and 1/3 by L-cysteine hydrochloride monohydrate/sodium bisulfite (w/w).

6. The liquid preparation according to claim 3, wherein a concentration of sodium bisulfite is in a range of from 0.02 to 0.30% (w/v).

7. The liquid preparation according to claim 3, wherein a quantity of sodium bisulfite is 1/35 to 3 times (weight ratio) relative to the active ingredient.

8. The liquid preparation according to claim 1, wherein the β-cyclodextrin derivative is β-cyclodextrin sulfobutyl ether sodium salt or hydroxypropyl β-cyclodextrin.

9. The liquid preparation according to claim 1, wherein a quantity of the β-cyclodextrin derivative relative to the active ingredient is such that a ratio between active ingredient: β-cyclodextrin derivative falls between 1:4 and 1:8 (molar ratio).

10. The liquid preparation according to claim 1, wherein a concentration of the β-cyclodextrin derivative is in a range of from 7.5 to 13.5% (w/v).

11. The liquid preparation according to claim 1, wherein the pH buffer is of amino acid buffer system or acetic acid buffer system.

12. The liquid preparation according to claim 11, wherein the amino acid buffer system comprises glycine or L-histidine.

13. The liquid preparation according to claim 12, wherein the amino acid buffer system comprises glycine with a concentration thereof in a range of from 50 to 150 mM.

14. The liquid preparation according to claim 1, wherein the pH value thereof is adjusted with a pH adjuster which is at least one selected from the group consisting of hydrochloric acid, methanesulfonic acid, acetic acid and sodium hydroxide.

15. The liquid preparation according to claim 14, wherein the pH adjuster is hydrochloric acid.

16. The liquid preparation according to claim 1, being prepared using water in which dissolved oxygen is reduced by inert gas replacement.

17. The liquid preparation according to claim 1, wherein a concentration of dissolved oxygen is less than or equal to 4 ppm.

18. The liquid preparation according to claim 1, further comprising a substance that reduces surface activity derived from the active ingredient.

19. The liquid preparation according to claim 18, wherein the substance that reduces the surface activity is at least one selected from the group consisting of sodium chloride, meglumine, L-arginine, glycine, L-histidine, L-glutamate, β-cyclodextrin derivative, propylene glycol, ethanol and chlorobutanol.

20. The liquid preparation according to claim 1, further comprising a substance that prevents formation of insoluble aggregates with free base of (2S)-1-(4-amino-2,3,5-trimethyl-phenoxy)-3-{4-[4-(4-fluorobenzyl)phenyl]-1-piperazinyl}-2-propanol.

21. The liquid preparation according to claim 20, wherein the substance that regulates formation of insoluble aggregates is at least one selected from the group consisting of methane sulfonic acid, hydrochloric acid and sodium chloride.

22. A liquid preparation comprising: 1 to 5 mg/mL of (2S)-1-(4-amino-2,3,5-trimethylphenoxy)-3-{4-[4-(4-fluorobenzyl)phenyl]-1-piperazinyl}-2-propanol or its pharmaceutically acceptable salt; 0.05 to 0.10% (w/v) of sodium bisulfite; 0.015 to 0.03% (w/v) of L-cysteine hydrochloride monohydrate; 7.5 to 10% (w/v) of β-cyclodextrin sulfobutyl ether sodium salt; 50 to 100 mM of glycine; and hydrochloric acid, wherein a pH value thereof falls within a range of from 3.0 to 4.5.

23. A liquid preparation comprising the liquid preparation according to claim 1 filled into a glass container having an inner wall treated with SiO₂ glass film at a liquid contacting side thereof.

24. A method of producing a liquid preparation, the method comprising the steps of:
(a) dissolving (2S)-1-(4-amino-2,3,5-trimethylphenoxy)-3-{4-[4-(4-fluorobenzyl)phenyl]-1-piperazinyl}-2-propanol or its pharmaceutically acceptable salt into water in which dissolved oxygen is reduced by inert gas replacement to prepare a solution of drug substance;
(b) dissolving a β-cyclodextrin derivative, a pH buffer and a pH adjuster into water and adjusting a pH value thereof to fall within a range of from 3 to 5, replacing dissolved oxygen by inert gas replacement, and dissolving at least one selected from the group consisting of sulfite, bisulfite, pyrosulfite, α-thioglycerol and cysteine to prepare a solution of inactive ingredients; and
(c) adding the solution of drug substance to the solution of inactive ingredients under inert gas flow and mixing the same.
